(19) United States
(12) Patent Application Publication
(10) Pub. No.: US 2024/0382469 A1
(43) Pub. Date: Nov. 21, 2024

(54) COMBINATION THERAPY WITH DEUTERATED IMIDAZOQUINOLINES

(71) Applicant: BIRDIE BIOPHARMACEUTICALS, INC., Wan Chai, Hong Kong (CN)

(72) Inventors: Pei-Jou LEE, Hsinchu City (TW); Yi-Tsui CHIU, Zhubei City (TW); Fu-Tan HSIEH, New Taipei City (TW)

(21) Appl. No.: 18/692,626

(22) PCT Filed: Sep. 16, 2022
(86) PCT No.: PCT/IB2022/000522

§ 371 Date: Mar. 15, 2024

Related U.S. Application Data
(60) Provisional application No. 63/245,694, filed on Sep. 17, 2021.

(51) Int. Cl. A61K 31/437; A61P 35/00; C07K 16/28

(52) U.S. Cl. CPC A61K 31/437; A61P 35/00; C07K 16/2818

(57) ABSTRACT

The present specification provides therapeutic combinations and methods for treating cancers using combination therapy.

### RELATED APPLICATIONS

This application claims priority of U.S. provisional patent application No. 63/245,694, filed Sep. 17, 2021, the entire content of which is incorporated herein by reference.

### FIELD

The present disclosure relates to combination immunotherapy and methods for treating cancer by using deuterated imidazoquinolines in combination with therapeutic antibody drugs.

### BACKGROUND

Resiquimod (R-848), belonging to imidazoquinoline compound, is a Toll-like receptor 7 and/or 8 (TLR7/8) agonist that acts as an immune response modulator, and has been reported having antiviral and antitumor activity. Also, resiquimod was used as a topical drug for viral skin lesions and skin cancer such as actinic keratosis (AK) (Meyer et al. (2013), Expert Opin Investig Drugs, 22(1):149-159). Resiquimod has not been approved yet for commercial use because of its dose limited toxicity. To solve problems of drug rapid metabolism, several deuterated analogues of resiquimod have been developed and exhibited an improved pharmacokinetic property (US2020/0207757A1). However, these deuterated analogues still have uncertainties about their therapeutic effect.

Therapeutic antibody drugs have been quickly developed in preclinical and clinical trials in the past decade. Among these, several antibody drugs targeting on different tumor antigens or surface molecules have been approved, e.g., PD-1 specific antibody such as Keytruda® (pembrolizumab), or Her2 specific antibody such as Herceptin® (trastuzumab). However, drug resistance or tolerance of long-term use is a major clinical problem for these antibody therapies. For example, some patients have been reported who can't benefit from continuous anti-PD-1/PD-L1 therapy due to resistance (Lei et al. (2020), Frontiers in Cell and Developmental Biology, 8:672). Therefore, there is still a need to develop a new therapy for treating cancer with improved efficacy and a long-term effect.

### SUMMARY

In general, the present disclosure directs to therapeutic combinations, pharmaceutical compositions and methods for treatment of cancers.

In one aspect, the disclosure relates to a combination comprising:
a. an effective amount of a targeted therapeutic against cancer; and
b. an effective amount of an immunotherapeutic that has a structure of Formula (I):

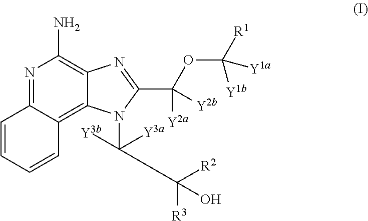

or a pharmaceutically acceptable salt thereof,
wherein
each of R¹, R² and R³ is independently selected from —CH₃, —CH₂D, —CHD₂, and —CD₃;
each of Y^{1a}, Y^{1b}, Y^{2a}, Y^{2b}, Y^{3a} and Y^{3b} is independently selected from hydrogen and deuterium; and
when each of R¹, R² and R³ is —CH₃, at least one of Y^{1a}, Y^{1b}, Y^{2a}, Y^{2b}, Y^{3a} and Y^{3b} is deuterium.

In some embodiments, said targeted therapeutic is capable of binding to a tumor antigen specifically or preferably in comparison to a non-tumor antigen, wherein said tumor antigen is selected from the group consisting of: 5T4, AGS-5, AGS-16, Angiopoietin 2, B7.1, B7.2, B7DC, B7H1, B7H2, B7H3, BT-062, BTLA, CAIX, Carcinoembryonic antigen, CTLA4, Cripto, ED-B, ErbB1, ErbB2, ErbB3, ErbB4, EGFL7, EpCAM, EphA2, EphA3, EphB2, FAP, Fibronectin, Folate Receptor, Ganglioside GM3, GD2, glucocorticoid-induced tumor necrosis factor receptor (GITR), gp100, gpA33, GPNMB, ICOS, IGF1R, Integrin αv, Integrin αvβ, KIR, LAG3, Lewis Y, Mesothelin, c-MET, MN Carbonic anhydrase IX, MUC1, MUC16, Nectin-4, NKGD2, NOTCH, OX40, OX40L, PD-1, PD-L1, PSCA, PSMA, RANKL, ROR1, ROR2, SLC44A4, Syndecan-1, TACI, TAG-72, Tenascin, TIM3, TRAILR1, TRAILR2, VEGFR-1, VEGFR-2, VEGFR-3, TIGIT, ILT2, ILT4, SIRPα, TROP2, Claudin18.2, CEACAM5, CD2, CD19, CD20, CD22, CD27, CD33, CD37, CD38, CD40, CD44, CD47, CD52, CD56, CD70, CD73, CD79, CD133, CD137, CD206 and variants thereof.

In another aspect, the disclosure relates to a pharmaceutical composition comprising the above combination and a pharmaceutical acceptable carrier.

In another aspect, the disclosure relates to a method for treating a tumor or abnormal cell proliferation in a subject that is in need of such treatment, comprising administering to said subject the above combination, or a pharmaceutical composition comprising the above combination.

In another aspect, the disclosure relates to a kit comprising the above combination.

### BRIEF DESCRIPTION OF DRAWINGS

FIG. 1 is a graph showing tumor size in each animal group treated or untreated with different deuterated compounds 1-3 in combination with an anti-PD-1 antibody.

FIG. 2 is a graph showing tumor size in each animal group treated or untreated with the combination of compound 1 and an anti-PD-1 antibody, or the combination of a proto-resiquimod and an anti-PD-1 antibody.

FIG. 3 is a graph showing tumor size in each animal group treated or untreated with compound 1 in combination with various therapeutic antibodies.

### DETAILED DESCRIPTION

Unless otherwise defined, all technical and scientific terms used herein have the same meaning as commonly understood by one of ordinary skill in the art to which this disclosure belongs. Furthermore, the following definitions are set forth to illustrate and define the meaning and scope of the various terms used to describe the disclosure.

### Definitions

Certain terms, whether used alone or as part of a phrase or another term, are defined below.

The articles “a” and “an” refer to one or to more than one of the grammatical object of the article.

Numerical values relating to measurements are subject to measurement errors that place limits on their accuracy. For this reason, all numerical values provided herein, unless otherwise indicated, are to be understood as being modified by the term “about.” Accordingly, the last decimal place of a numerical value provided herein indicates its degree of accuracy. Where no other error margins are given, the maximum margin is ascertained by applying the rounding-off convention to the last decimal place or last significant digit when a decimal is not present in the given numerical value.

The term “amelioration” means a lessening of severity of at least one indicator of a condition or disease, such as a delay or slowing in the progression of one or more indicators of a condition or disease. The severity of indicators may be determined by subjective or objective measures which are known to those skilled in the art.

The terms “composition” and “pharmaceutical composition” refer to a mixture of at least one compound described herein with a pharmaceutically acceptable carrier. The pharmaceutical composition facilitates administration of the compound to a patient or subject. Multiple techniques of administering a compound exist including, but not limited to, intravenous, oral, aerosol, parenteral, ophthalmic, pulmonary, and topical administration.

The term “compound,” when referring to a compound of this disclosure, refers to a collection of molecules having an identical chemical structure, except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of this disclosure will depend upon a number of factors including the isotopic purity of deuterated reagents used to make the compound and the efficiency of incorporation of deuterium in the various synthesis steps used to prepare the compound. However, as set forth above the relative amount of such isotopologues in total will be less than 49.9% of the compound. In other embodiments, the relative amount of such isotopologues in total will be less than 47.5%, less than 40%, less than 32.5%, less than 25%, less than 17.5%, less than 10%, less than 5%, less than 3%, less than 1%, or less than 0.5% of the compound.

The terms “effective amount” and “therapeutically effective amount” refer to an amount of therapeutic compound, such as a compound described herein, administered to a subject, either as a single dose or as part of a series of doses, which is effective to produce a desired therapeutic effect. In general, the therapeutically effective amount can be estimated initially either in cell culture assays or in mammalian animal models, for example, in non-human primates, mice, rabbits, dogs, or pigs. The animal model may also be used to determine the appropriate concentration range and route of administration. Such information can then be used to determine useful doses and routes for administration in non-human subjects and human subjects.

The term “isotopic enrichment factor” as used herein means the ratio between the isotopic abundance and the natural abundance of a specified isotope. In various embodiments, compounds of this disclosure have an isotopic enrichment factor for each designated deuterium atom of at least 3340 (50.1% deuterium incorporation at each designated deuterium atom), at least 3500 (52.5% deuterium incorporation), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

The term “isotopologue” refers to a species that differs from a specific compound of this disclosure only in the isotopic composition thereof.

The term “pharmaceutically acceptable carrier” means a pharmaceutically acceptable material, composition or carrier, such as a liquid filler, solid filler, stabilizer, dispersing agent, suspending agent, diluent, excipient, thickening agent, solvent, or encapsulating material, involved in carrying or transporting at least one compound described herein within or to the patient such that the compound may perform its intended function. A given carrier must be “acceptable” in the sense of being compatible with the other ingredients of a particular formulation, including the compounds described herein, and not injurious to the patient. Other ingredients that may be included in the pharmaceutical compositions described herein are known in the art and described, for example, in “Remington's Pharmaceutical Sciences” (Genaro (Ed.), Mack Publishing Co., 1985), the entire content of which is incorporated herein by reference.

The term “pharmaceutically acceptable salt” refers to derivatives of the disclosed compounds wherein the parent compound is modified by converting an existing acid or base moiety to its salt form. Pharmaceutically acceptable salts can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two solvents. Lists of suitable salts are found in “Handbook of Pharmaceutical Salts: Properties, Selection, and Use” (P. Henrich Stahl & Camille G. Wermuth (Eds.), VHCA & Wiley-VCH, 2002), the entire content of which is incorporated herein by reference.

The term “refractory disease” refers to a disease that continues to progress during treatment with a pharmaceutical ingredient other than the compounds provided herein, partially responds to the other treatment, or transiently responds to the other treatment. The term may be applied to each of the diseases referred to herein.

The term “substituted” refers to replacement of hydrogen attached to another group with an atom or group of atoms as the replacement substituent, wherein each substituent is independently selected.

The terms “treatment” or “treating” refer to the application of one or more specific procedures used for the amelioration of a disease. A “prophylactic” treatment, refers to reducing the rate of progression of the disease or condition being treated, delaying the onset of that disease or condition, or reducing the severity of its onset.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the described subject matter and does not pose a limitation on the scope of the subject matter otherwise claimed. No language in the specification should be construed as indicating any non-claimed element essential to practicing the described subject matter.

Groupings of alternative elements or embodiments of this disclosure are not to be construed as limitations. Each group member may be referred to and claimed individually or in any combination with other members of the group or other elements found herein. Furthermore, a recited member of a group may be included in, or excluded from, another recited group for reasons of convenience or patentability. When any such inclusion or exclusion occurs, the specification is deemed to contain the group as modified thus fulfilling the written description of all Markush groups used in the appended claims.

References have been made to patents and printed publications throughout this specification, each of which are individually incorporated herein by reference in their entirety.

It is to be understood that the embodiments of this disclosure are illustrative. Accordingly, the present disclosure is not limited to that precisely as shown and described.

It will be recognized that some variation of natural isotopic abundance occurs in a synthesized compound depending upon the origin of chemical materials used in the synthesis. Thus, a synthesized compound will inherently contain small amounts of deuterated isotopologues. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Unless otherwise stated, when a position is designated specifically as “D” or “deuterium”, the position is understood to have deuterium at an abundance that is at least 3340 times greater than the natural abundance of deuterium, which is 0.015% (i.e., at least 50.1% incorporation of deuterium).

Generally, the present disclosure provides therapeutic combinations, pharmaceutical compositions and methods for treatment of cancers.

In one aspect, the disclosure relates to a combination, comprising:
a. an effective amount of a targeted therapeutic against cancer; and
b. an effective amount of an immunotherapeutic that is a TLR 7 and/or 8 agonist and has a structure of Formula (I):

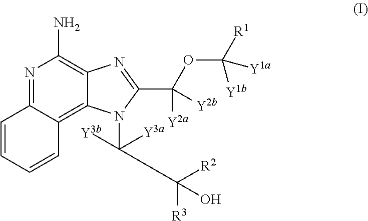

or a pharmaceutically acceptable salt thereof,
wherein
each of R¹, R² and R³ is independently selected from —CH₃, —CH₂D, —CHD₂, and —CD₃;
each of Y^{1a}, Y^{1b}, Y^{2a}, Y^{2b}, Y^{3a} and Y^{3b} is independently selected from hydrogen and deuterium; and
when each of R¹, R² and R³ is —CH₃, at least one of Y^{1a}, Y^{1b}, Y^{2a}, Y^{2b}, Y^{3a} and Y^{3b} is deuterium.

In particular, each position that is designated as containing deuterium has at least 50.1% deuterium incorporation at that position.

In one embodiment of the disclosure, each of R¹, R² and R³ is independently selected from —CH₃ and —CD₃. In some embodiments, R¹ is —CD₃. In some embodiments, R¹ is —CH₃. In some embodiments, R² is —CD₃. In some embodiments, R² is —CH₃. In some embodiments, R³ is —CD₃. In some embodiments, R³ is —CH₃.

In another embodiment, each of R² and R³ is —CD₃, or each of R² and R³ is —CH₃.

In some embodiments, Y^{1a} and Y^{1b} are the same; Y^{2a} and Y^{2b} are the same; and Y^{3a} and Y^{3b} are the same.

In some embodiments, R¹ is —CD₃, and each of Y^{1a} and Y^{1b} is deuterium.

In some embodiments, each of R² and R³ is —CD₃, and each of Y^{3a} and Y^{3b} is deuterium.

In some embodiments, Y^{1a} and Y^{1b} are the same; Y^{2a} and Y^{2b} are the same; Y^{3a} and Y^{3b} are the same; and the values of R¹, R², R³, Y^{1a}, Y^{1b}, Y^{2a}, Y^{2b}, Y^{3a} and Y^{3b} are selected from the values listed in Table 1 below. For the compounds defined by the values of the variables of formula (I) listed in Table 1, any atom not designated as D (deuterium) is present at its natural isotopic abundance.

TABLE 1

| Compound | R¹ | R² | R³ | Y^{1a}/Y^{1b} | Y^{2a}/Y^{2b} | Y^{3a}/Y^{3b} |
|---|---|---|---|---|---|---|
| 1 | CD₃ | CH₃ | CH₃ | D | H | H |
| 2 | CH₃ | CD₃ | CD₃ | H | H | D |
| 3 | CH₃ | CD₃ | CD₃ | H | H | H |
| 4 | CD₃ | CH₃ | CH₃ | H | D | H |
| 5 | CD₃ | CH₃ | CH₃ | D | D | H |
| 6 | CH₃ | CH₃ | CH₃ | D | D | H |
| 7 | CH₃ | CH₃ | CH₃ | D | H | H |
| 8 | CH₃ | CH₃ | CH₃ | H | D | H |
| 9 | CH₃ | CD₃ | CH₃ | H | H | D |
| 10 | CH₃ | CH₃ | CD₃ | H | H | D |
| 11 | CH₃ | CD₃ | CH₃ | H | H | H |
| 12 | CH₃ | CH₃ | CD₃ | H | H | H |
| 13 | CH₃ | CH₃ | CH₃ | H | H | D |
| 14 | CD₃ | CH₃ | CH₃ | H | H | H |
| 15 | CH₃ | CH₃ | CH₃ | D | H | D |
| 16 | CH₃ | CD₃ | CD₃ | D | H | H |
| 17 | CH₃ | CD₃ | CH₃ | D | H | H |
| 18 | CH₃ | CH₃ | CD₃ | D | H | H |
| 19 | CH₃ | CH₃ | CH₃ | H | D | D |
| 20 | CH₃ | CD₃ | CD₃ | H | D | H |
| 21 | CH₃ | CD₃ | CH₃ | H | D | H |
| 22 | CH₃ | CH₃ | CD₃ | H | D | H |
| 23 | CD₃ | CH₃ | CH₃ | H | H | D |
| 24 | CD₃ | CD₃ | CD₃ | H | H | H |
| 25 | CD₃ | CD₃ | CH₃ | H | H | H |
| 26 | CD₃ | CH₃ | CD₃ | H | H | H |
| 27 | CH₃ | CH₃ | CH₃ | D | D | D |
| 28 | CH₃ | CD₃ | CD₃ | D | D | H |
| 29 | CH₃ | CD₃ | CH₃ | D | D | H |
| 30 | CH₃ | CH₃ | CD₃ | D | D | H |
| 31 | CD₃ | CH₃ | CH₃ | D | H | D |
| 32 | CH₃ | CD₃ | CD₃ | D | H | D |
| 33 | CH₃ | CD₃ | CH₃ | D | H | D |
| 34 | CH₃ | CH₃ | CD₃ | D | H | D |
| 35 | CD₃ | CD₃ | CD₃ | D | H | H |
| 36 | CD₃ | CD₃ | CH₃ | D | H | H |
| 37 | CD₃ | CH₃ | CD₃ | D | H | H |
| 38 | CD₃ | CH₃ | CH₃ | H | D | D |
| 39 | CH₃ | CD₃ | CD₃ | H | D | D |
| 40 | CH₃ | CD₃ | CH₃ | H | D | D |
| 41 | CH₃ | CH₃ | CD₃ | H | D | D |
| 42 | CD₃ | CD₃ | CD₃ | H | D | H |
| 43 | CD₃ | CD₃ | CH₃ | H | D | H |
| 44 | CD₃ | CH₃ | CD₃ | H | D | H |
| 45 | CD₃ | CD₃ | CD₃ | H | H | D |
| 46 | CD₃ | CD₃ | CH₃ | H | H | D |
| 47 | CD₃ | CH₃ | CD₃ | H | H | D |
| 48 | CD₃ | CH₃ | CH₃ | D | D | D |
| 49 | CH₃ | CD₃ | CD₃ | D | D | D |
| 50 | CH₃ | CD₃ | CH₃ | D | D | D |
| 51 | CH₃ | CH₃ | CD₃ | D | D | D |
| 52 | CD₃ | CD₃ | CD₃ | D | D | H |
| 53 | CD₃ | CD₃ | CH₃ | D | D | H |
| 54 | CD₃ | CH₃ | CD₃ | D | D | H |
| 55 | CD₃ | CD₃ | CD₃ | D | H | D |
| 56 | CD₃ | CD₃ | CH₃ | D | H | D |
| 57 | CD₃ | CH₃ | CD₃ | D | H | D |
| 58 | CD₃ | CD₃ | CD₃ | H | D | D |
| 59 | CD₃ | CD₃ | CH₃ | H | D | D |
| 60 | CD₃ | CH₃ | CD₃ | H | D | D |
| 61 | CD₃ | CD₃ | CD₃ | D | D | D |
| 62 | CD₃ | CD₃ | CH₃ | D | D | D |
| 63 | CD₃ | CH₃ | CD₃ | D | D | D |

In another embodiment, an immunotherapeutic of formula (I) is selected from the group consisting of:

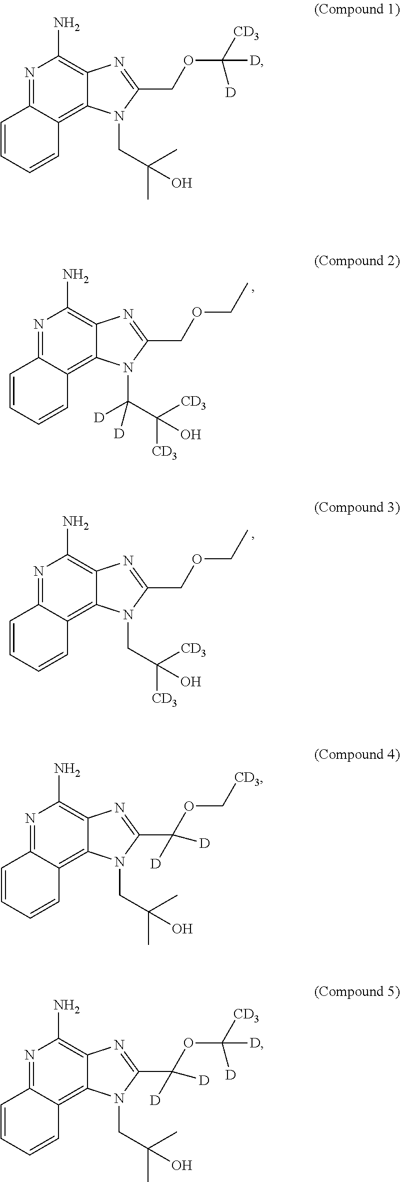

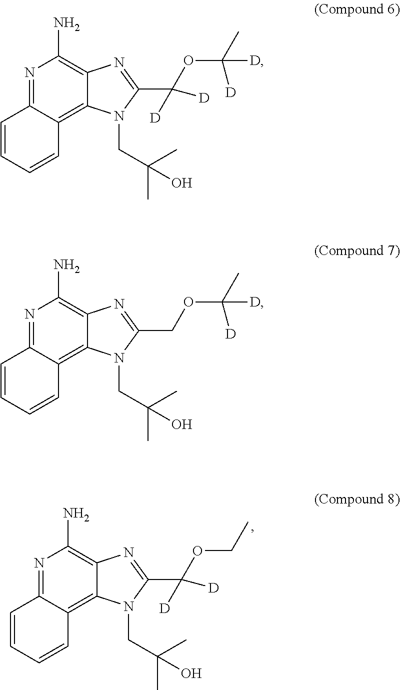

or a pharmaceutically acceptable salt thereof.

In another embodiment, an immunotherapeutic of formula (I) is selected from the group consisting of:

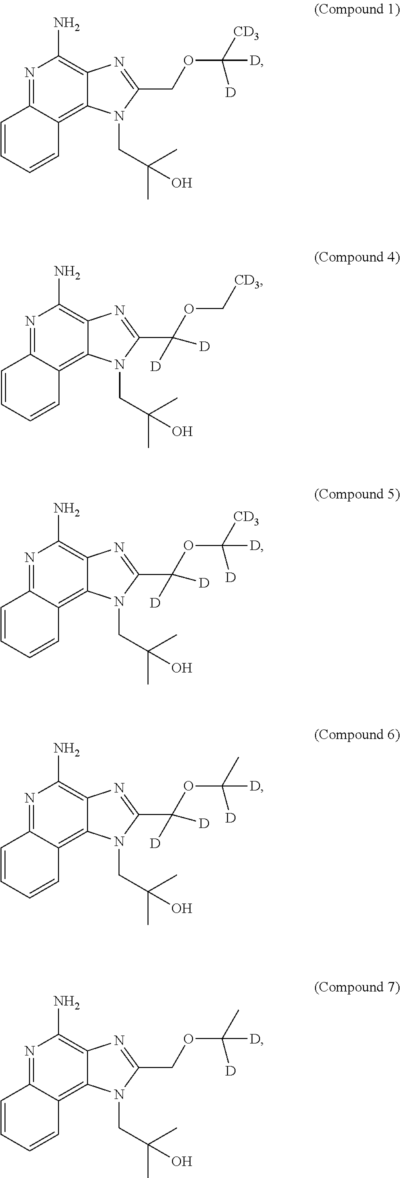

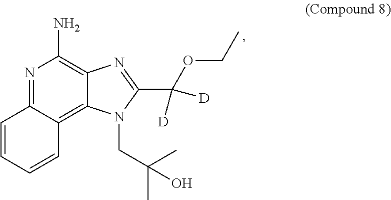 [SMILES: [2H]C([2H])(OCC)c1nc2c(N)nc3ccccc3c2n1CC(C)(C)O]

or a pharmaceutically acceptable salt thereof.

In another embodiment, an immunotherapeutic of formula (I) is selected from the group consisting of:

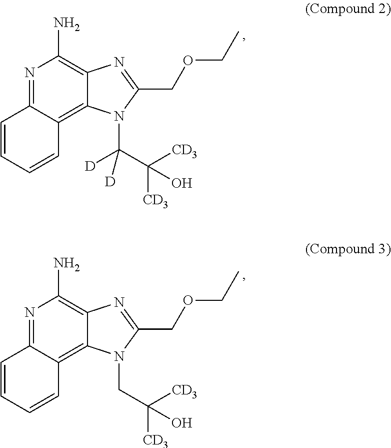 [SMILES: [2H]C([2H])([2H])C(O)(C([2H])([2H])[2H])C([2H])([2H])n1c(COCC)nc2c(N)nc3ccccc3c21 ; [2H]C([2H])([2H])C(O)(Cn1c(COCC)nc2c(N)nc3ccccc3c21)C([2H])([2H])[2H]]

or a pharmaceutically acceptable salt thereof.

In one embodiment, the immunotherapeutic is:

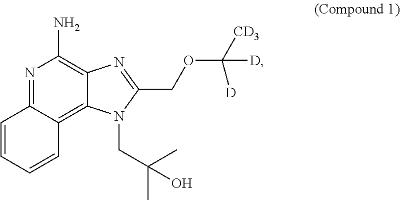

or a pharmaceutically acceptable salt thereof.

In one embodiment, the immunotherapeutic is:

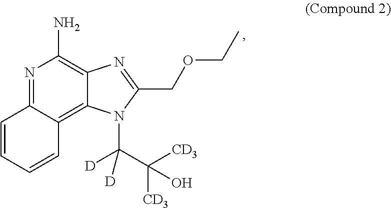 [SMILES: [2H]C([2H])([2H])C(O)(C([2H])([2H])[2H])C([2H])([2H])n1c(COCC)nc2c(N)nc3ccccc3c21]

or a pharmaceutically acceptable salt thereof.

In one embodiment, the immunotherapeutic is:

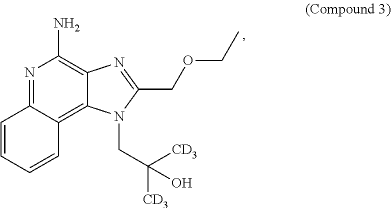 [SMILES: [2H]C([2H])([2H])C(O)(Cn1c(COCC)nc2c(N)nc3ccccc3c21)C([2H])([2H])[2H]]

or a pharmaceutically acceptable salt thereof.

The immunotherapeutic of formula (I) can be synthesized according the manufacture method as described in US patent 2020/0207757 A1, which is incorporated herein by reference. The exemplary compounds 1-3 used in the following Example 1 are also synthesized according the manufacture method as described in the above patent.

In one embodiment, the targeted therapeutic is capable of binding to a tumor cell specifically or preferably in comparison to a non-tumor cell, wherein said tumor cell is of a carcinoma, a sarcoma, a lymphoma, a myeloma, or a central nervous system cancer.

In one embodiment, the targeted therapeutic is capable of binding to a tumor antigen specifically or preferably in comparison to a non-tumor antigen, wherein said tumor antigen is selected from the group consisting of: 5T4, AGS-5, AGS-16, Angiopoietin 2, B7.1, B7.2, B7DC, B7H1, B7H2, B7H3, BT-062, BTLA, CAIX, Carcinoembryonic antigen, CTLA4, Cripto, ED-B, ErbB1 (HER1/EGFR), ErbB2 (HER2/neu), ErbB3, ErbB4, EGFL7, EpCAM, EphA2, EphA3, EphB2, FAP, Fibronectin, Folate Receptor, Ganglioside GM3, GD2, glucocorticoid-induced tumor necrosis factor receptor (GITR), gp100, gpA33, GPNMB, ICOS, IGF1R, Integrin αv, Integrin αvβ, KIR, LAG3, Lewis Y, Mesothelin, c-MET, MN Carbonic anhydrase IX, MUC1, MUC16, Nectin-4, NKGD2, NOTCH, OX40, OX40L, PD-1, PD-L1, PSCA, PSMA, RANKL, ROR1, ROR2, SLC44A4, Syndecan-1, TACI, TAG-72, Tenascin, TIM3, TRAILR1, TRAILR2, VEGFR-1, VEGFR-2, VEGFR-3, TIGIT, ILT2, ILT4, SIRPα, TROP2, Claudin18.2, CEACAM5, CD2, CD19, CD20, CD22, CD27, CD33, CD37, CD38, CD40, CD44, CD47, CD52, CD56, CD70, CD73, CD79, CD133, CD137, CD206 and variants thereof.

In another embodiment, the tumor antigen is selected from the group consisting of: PD-1, PD-L1, CTLA4, ErbB1, ErbB2, TIGIT, OX40, LAG3, CD47, CD137 and variants thereof.

In another embodiment, the targeted therapeutic comprises an immunoglobulin, a protein, a peptide, a small molecule, a nanoparticle, or a nucleic acid.

In another embodiment, the targeted therapeutic comprises an antibody, or a functional fragment thereof.

In some embodiments, the antibody is selected from the group consisting of: Keytruda® (pembrolizumab), Opdivo® (nivolumab), Libtayo® (cemiplimab), Jemperli® (dostarlimab), Tecentriq® (atezolizumab), Imfinzi® (durvalumab), Bavencio® (avelumab), Yervoy® (ipilimumab), Tarceva® (erlotinib), Tagrisso® (osimertinib), Iressa® (gefitinib), Erbitux® (cetuximab), Vectibix® (panitumumab), Vizimpro® (dacomitinib), Tykerb® (lapatinib), Portrazza® (necitumumab), Caprelsa® (vandetanib), Nerlynx® (neratinib), Herceptin® (trastuzumab), Tukysa® (tucatinib), Perjeta® (pertuzumab), Rituxan® (rituximab), Arzerra® (Ofatumumab), Benlysta® (belimumab), Tremelimumab, Dacetuzumab, Urelumab, MPDL3280A, Lambrolizumab, and Blinatumomab, CT-011, BMS-936559, MPDL3280A, MED14736, MSB0010718C, tiragolumab, PF-04518600, BMS-986178, MEDI6469, Relatlimab (BMS-986016), AK117, IBI188, STI-6643, LVGN6051, Urelumab (BMS-663513), FS222, FS120.

In another embodiment, the targeted therapeutic comprises a Fab, Fab′, F(ab′)2, single domain antibody, T and Abs dimer, Fv, scFv, dsFv, ds-scFv, Fd, linear antibody, minibody, diabody, bispecific antibody fragment, bibody, tribody, sc-diabody, kappa (lamda) body, BiTE, DVD-Ig, SIP, SMIP, DART, or an antibody analogue comprising one or more CDRs.

In another aspect, the disclosure also relates to a pharmaceutical composition comprising:
a. the combination according to the disclosure; and
b. a pharmaceutical acceptable carrier/excipient.

The pharmaceutical composition may be an enteral or a parenteral dosage form, suitable for transdermal, transmucosal, nasopharyngeal, pulmonary or direct injection, or for systemic (e.g., parenteral) or local (e.g., intratumor or intralesional injection) administration. Parenteral injection may be via intravenous, intraperitoneal, intramuscular, subcutaneous or intradermal routes.

The term “combination” or “therapeutic combination” refers to a combination of one or more active drug substances that have a therapeutic effect. A therapeutic combination may be provided in a single pharmaceutical composition so that both the targeted therapeutics and the immunotherapeutic can be administered together. In alternative embodiments, a therapeutic combination may be provided using more than one pharmaceutical composition. In such embodiments, a targeted therapeutic may be provided in one pharmaceutical composition and an immunotherapeutic may be provided in a second pharmaceutical composition so that the two compounds can be administered separately such as, for example, at different times, by different routes of administration, and the like. Thus, it also may be possible to provide the targeted therapeutic and the immunotherapeutic in different dosing regimens.

The term “pharmaceutically acceptable carrier/excipient” includes any and all solvents, dispersion media, coatings, surfactants, antioxidants, preservatives, isotonic agents, absorption delaying agents, salts, drugs, drug stabilizers, binders, excipients, disintegration agents, lubricants, sweetening agents, flavoring agents, dyes, and combinations thereof, as would be known to one of ordinary skill in the art (see, for example, Remington's Pharmaceutical Sciences, 18th Ed. Mack Printing Company, 1990,pp. 1289-1329, incorporated herein by reference). Except in so far as any conventional carrier is incompatible with the active ingredient, its use in the therapeutic or pharmaceutical compositions is contemplated.

In another aspect, the disclosure also relates to a method for treating a tumor or abnormal cell proliferation in a subject that is in need of such treatment, comprising administering to said subject the combination of the disclosure.

In some embodiments, administration in combination with one or more further therapeutic agents includes simultaneous and consecutive administration in any order.

For example, the combination containing the active ingredients, e.g., an immunotherapeutic and a targeted therapeutic, are (1) both administered to a patient simultaneously in the form of a single entity or dosage, or (2) administered to a patient as separate entities either simultaneously or sequentially. Such administration provides therapeutically effective amounts of the active ingredients in the body of the patient.

In some embodiments, the administration of each active ingredient can be independently selected from an intravenous route, an intraperitoneal route, an intramuscular route, an intratumor route, a subcutaneous route, or an intradermal route, so as to provide an effective treatment.

In one embodiment, the abnormal cell proliferation comprises a pre-cancerous lesion. In another embodiment, the abnormal proliferation is of cancer cells.

In one embodiment, the cancer is selected from the group consisting of: cholangiocarcinoma, breast cancer, colorectal cancer, diffuse large B-cell lymphoma, endometrial cancer, follicular lymphoma, gastric cancer, glioblastoma, head and neck cancer, hepatocellular cancer, non-small cell lung cancer, small cell lung cancer, melanoma, multiple myeloma, ovarian cancer, cervical cancer, pancreatic cancer, prostate cancer, and renal cell carcinoma.

In another embodiment, the method of treating comprises administering to said subject a formulation comprising said immunotherapeutic in a dose of between about 0.0005 mg/kg, 0.0006 mg/kg, 0.0007 mg/kg, 0.0008 mg/kg, 0.0009 mg/kg, 0.001 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, 0.01 mg/kg, 0.02 mg/kg, 0.025 mg/kg, 0.05 mg/kg, 0.075 mg/kg, 0.1 mg/kg, 0.125 mg/kg, 0.15 mg/kg, 0.175 mg/kg, 0.2 mg/kg, 0.215 mg/kg, 0.25 mg/kg, 0.5 mg/kg, 0.75 mg/kg, 1 mg/kg, 1.25 mg/kg, 1.5 mg/kg, 1.75 mg/kg, 2 mg/kg, 2.25 mg/kg, to about 2.5 mg/kg all inclusive, twice daily, once daily, once every two, three, four, five or six days, or once, twice, or three times per week.

In another embodiment, the method of treating comprises administering to said subject a formulation comprising said immunotherapeutic in a dose of between about 0.0005 mg/kg, to about 0.0006 mg/kg, 0.0007 mg/kg, 0.0008 mg/kg, 0.0009 mg/kg, 0.001 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, 0.01 mg/kg, 0.02 mg/kg, 0.025 mg/kg, 0.05 mg/kg, 0.075 mg/kg, 0.1 mg/kg, 0.125 mg/kg, 0.15 mg/kg, 0.175 mg/kg, 0.2 mg/kg, 0.215 mg/kg, 0.25 mg/kg, 0.5 mg/kg, 0.75 mg/kg, 1 mg/kg, 1.25 mg/kg, 1.5 mg/kg, 1.75 mg/kg, 2 mg/kg, 2.25 mg/kg, or 2.5 mg/kg all inclusive, twice daily, once daily, once every two, three, four, five or six days, or once, twice, or three times per week.

In another embodiment, the method of treating comprises administering to said subject a formulation comprising said immunotherapeutic in a dose of less than or about 0.0005 mg/kg, 0.0006 mg/kg, 0.0007 mg/kg, 0.0008 mg/kg, 0.0009 mg/kg, 0.001 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, 0.01 mg/kg, 0.02 mg/kg, 0.025 mg/kg, 0.05 mg/kg, 0.075 mg/kg, 0.1 mg/kg, 0.125 mg/kg, 0.15 mg/kg, 0.175 mg/kg, 0.2 mg/kg, 0.215 mg/kg, 0.25 mg/kg, 0.5 mg/kg, 0.75 mg/kg, 1 mg/kg, 1.25 mg/kg, 1.5 mg/kg, 1.75 mg/kg, 2 mg/kg, 2.25 mg/kg, or 2.5 mg/kg, twice daily, once daily, once every two, three, four, five or six days, or once, twice, or three times per week.

In another embodiment, the immunotherapeutic in said subject has a local concentration that is between about 0.005 μg/ml to about 12 μg/ml.

In another embodiment, the said immunotherapeutic in said subject has a local concentration that is between about 0.05 μg/ml, 0.1 μg/ml, 0.15 μg/ml, 0.2 μg/ml, 0.3 μg/ml, or 0.4 μg/ml, to about 0.5 μg/ml.

In another aspect, the present disclosure provides kits containing the therapeutic combinations provided herein and directions for using the therapeutic combinations. The kit may also include a container and optionally one or more vial, test tube, flask, bottle, or syringe. Other formats for kits will be apparent to those of skill in the art and are within the scope of the present disclosure.

### EXAMPLES

The following non-limiting examples are provided for illustrative purposes only in order to facilitate a more complete understanding of representative embodiments now contemplated. These examples should not be construed to limit any of the embodiments described in the present specification.

### Antibody

The antibodies used herein were purchased from Bio X Cell, Inc., including anti-PD-1 antibody (catalog no. BE0146), anti-CD137 antibody (catalog no. BE0239), anti-TIGIT antibody (catalog no. BE0274), anti-OX40 antibody (catalog no. BE0031), anti-LAG3 antibody (catalog no. BE0174), and anti-CD47 antibody (catalog no. BE0270).

### Example 1: In Vivo Efficacy Analysis on Different Deuterated Compounds in Combination With an Anti-PD-1 Antibody in CT26 Animal Model.

Female BALB/c mice (5 to 6-week-old) were randomly divided into 5 groups (n=5 per group), including (1) placebo (PBS; s.c.); (2) anti-PD-1 antibody (50 μg; i.p.); (3) compound 1 and anti-PD-1 antibody; (4) compound 2 and anti-PD-1 antibody; (5) compound 3 and anti-PD-1 antibody.

To challenge mice, CT26 tumor cells (1×10⁶ in 0.1 mL) were injected subcutaneously on day 0. Then, mice in each group received totally three doses of placebo, anti-PD-1 antibody or indicated combination therapy on days 6, 9 and 12. In each dose of combination therapy, 1.6 μg of TLR7/8 agonist (compound 1, 2 or 3) was given subcutaneously, and 50 μg of anti-PD-1 antibody (mAb) was given intraperitoneally. All mice were sacrificed on day 28. The tumor size was determined twice a week by multiplication of caliper measurements based on the modified ellipsoidal formula: Tumor volume=½ (length × width²). Mice with tumor length over 2 cm were considered dead and mice without measurable or palpable tumor masses were considered tumor-free. The significance of all comparisons was calculated by using t-test, and results considered significant when p<0.05.

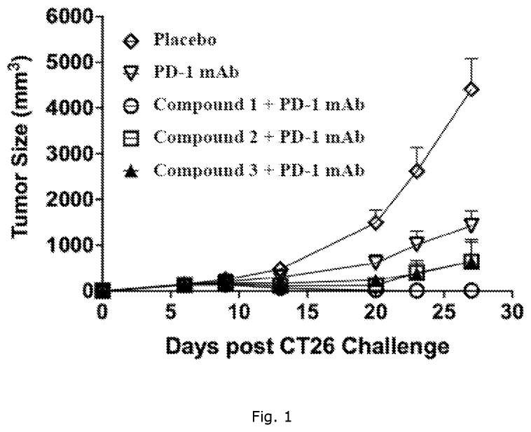

FIG. 1 showed the tumor size in each animal group treated with different deuterated compounds 1-3 in combination with an anti-PD-1 antibody. The results indicated that anti-PD-1 antibody could moderately reduce tumor size in CT26 tumor model, and the combinations of deuterated compound (compound 1, 2 or 3) and anti-PD-1 antibody all could significantly reduce tumor size as compared to placebo group (p<0.05), and even as compared to anti-PD-1 antibody alone group (p<0.05). Among that, the combination of compound 1 and anti-PD-1 antibody almost completely suppressed tumor size during the entire experimental period. In addition, while in combination with an anti-PD-1 antibody, compound 1 surprisingly exhibited an outstanding and significant tumor inhibitory effect than compounds 2 or 3 (p<0.05 for each comparison). The tumors in groups of compounds 2 or 3 in combination with anti-PD-1 antibody were initially well controlled, however, slightly grew at the last week. The results indicate that the combination therapies of the disclosure can effectively suppress tumor growth. Especially, compound 1 in combination with a therapeutic antibody (e.g., anti-PD-1 antibody) showed an unexpected anti-tumor effect.

### Example 2: In Vivo Efficacy Analysis of Compound 1 or Proto-Resiquimod in Combination With an Anti-PD-1 Antibody in CT26 Animal Model.

Female BALB/c mice (5 to 6-week-old) were randomly divided into 4 groups (n=5 per group), including (1) placebo (PBS; i.p.); (2) anti-PD-1 antibody (50 μg; i.p.); (3) compound 1 (1.6 μg; s.c.) and anti-PD-1 antibody (50 μg; i.p.); (4) proto-resiquimod (1.6 μg; s.c.) and anti-PD-1 antibody (50 μg; i.p.).

To challenge mice, CT26 tumor cells (1×10⁶ in 0.1 mL) were injected subcutaneously on day 0. Then, mice in each group received totally three doses of placebo, anti-PD-1 antibody or indicated combination therapy. In placebo group, mice received PBS (s.c.) on days 7, 14 and 21. In anti-PD-1 antibody alone group, mice received 50 μg of anti-PD-1 antibody (i.p.) on days 7, 10 and 14. In each combination therapy group, mice received 1.6 μg of TLR7/8 agonist (compound 1 or proto-resiquimod) (s.c.) on days 7, 14 and 21, and 50 μg of anti-PD-1 antibody (i.p.) on days 7, 10 and 14. All mice were sacrificed on day 28. By using the same method as described in example 1, the tumor size was determined and the significance of all comparisons was calculated.

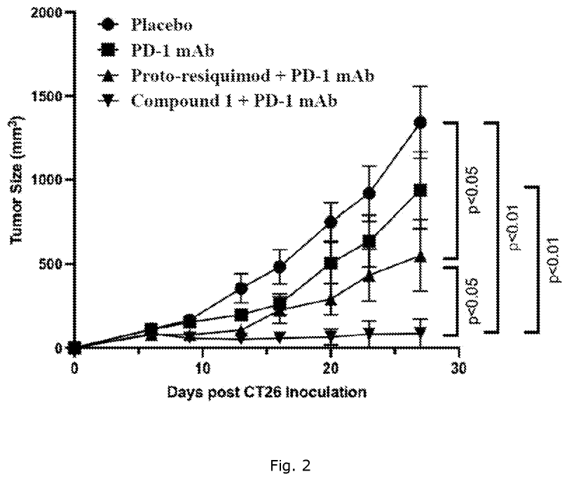

FIG. 2 showed the tumor size in each animal group treated with compound 1 or proto-resiquimod in combination with an anti-PD-1 antibody. The results indicated that anti-PD-1 antibody alone has limited effects in suppressing tumor size, the combination of proto-resiquimod and anti-PD-1 antibody could moderately suppress tumor size, and the combination of compound 1 and anti-PD-1 antibody could surprisingly and potently inhibit tumor size during the entire experimental period. Particularly, while in combination with an anti-PD-1 antibody, compound 1 exhibited a more significant tumor inhibitory effect than proto-resiquimod (p<0.05).

### Example 3: In Vivo Efficacy Analysis on Compound 1 in Combination With Various Therapeutic Antibodies in TC-1 Animal Model.

Female C57BL/6 mice (5 to 6-week-old) were randomly divided into 7 groups (n=5 per group), including (1) placebo (PBS, i.p.); (2) compound 1 (1.6 μg; s.c.); (3) compound 1 and anti-CD137 antibody; (4) compound 1 and anti-TIGIT antibody; (5) compound 1 and anti-OX40 antibody; (6) compound 1 and anti-LAG3 antibody; (7) compound 1 and anti-CD47 antibody.

To challenge, each mouse was subcutaneously injected with 1×10⁶ TC-1 cells in a volume of 0.1 mL into the left flank at day 0. Then, mice in each group received totally five doses of placebo, compound 1 or indicated combination therapy on days 9, 12, 15, 18 and 21. In each dose of combination therapy, 1.6 μg of compound 1 was given subcutaneously, and 200 μg of therapeutic antibody (anti-CD137, anti-TIGIT, anti-OX40, anti-LAG3 or anti-CD47 antibody) was given intraperitoneally. All mice were sacrificed on day 34. By using the same method as described in example 1, the tumor size was determined and the significance of all comparisons was calculated.

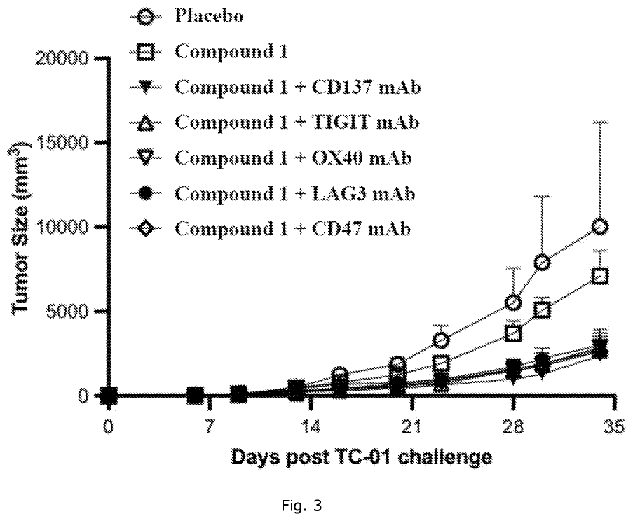

FIG. 3 showed the tumor size in each animal group treated with compound 1 in combination with various therapeutic antibody. The results indicated that compound 1 in combination with each therapeutic antibody, including anti-CD137, anti-TIGIT, anti-OX40, anti-LAG3 or anti-CD47 antibody, all could significantly suppress tumor size in TC-1 tumor model as compared to placebo group (p<0.05) or compound 1 alone group (p<0.05). The combination therapies of the disclosure all showed potent effects in suppressing tumor growth.

What is claimed is:

1. A combination, comprising:
(a) an effective amount of a targeted therapeutic against cancer; and
(b) an effective amount of an immunotherapeutic that has a structure of Formula (I): 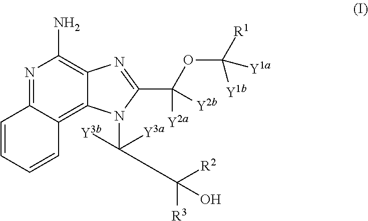
or a pharmaceutically acceptable salt thereof,
wherein
each of R¹, R² and R³ is, independently, —CH₃, —CH₂D, —CHD₂, or —CD₃;
each of Y^{1a}, Y^{1b}, Y^{2a}, Y^{2b}, Y^{3a} and Y^{3b} is, independently, hydrogen or deuterium; and
when each of R¹, R² and R³ is —CH₃, at least one of Y^{1a}, Y^{1b}, Y^{2a}, Y^{2b}, Y^{3a} and Y^{3b} is deuterium.

2. The combination of claim 1, wherein each of R¹, R² and R³ is, independently, —CH₃ or —CD₃.

3. The combination of claim 1, wherein R¹ is —CD₃.

4. The combination of claim 1, wherein R¹ is —CH₃.

5. The combination of claim 1, wherein each of R² and R³ is —CD₃.

6. The combination of claim 1, wherein each of R² and R³ is —CH₃.

7. The combination of claim 1, wherein Y^{1a} and Y^{1b} are the same; Y^{2a} and Y^{2b} are the same; and Y^{3a} and Y^{3b} are the same.

8. The combination of claim 1, wherein R¹ is —CD₃, and each of Y^{1a} and Y^{1b} is deuterium.

9. The combination of claim 1, wherein each of R² and R³ is —CD₃, and each of Y^{3a} and Y^{3b} is deuterium.

10. The combination of claim 1, wherein said immunotherapeutic is: 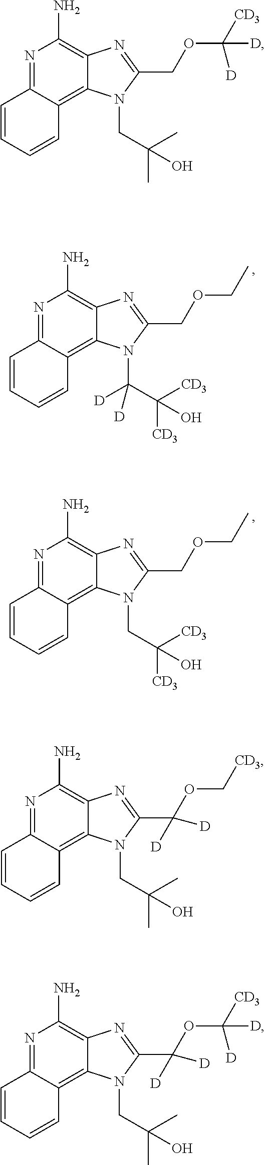 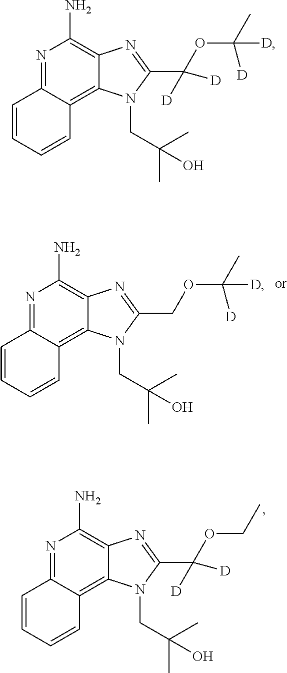
or a pharmaceutically acceptable salt thereof.

11. The combination of claim 10, wherein said immunotherapeutic is: 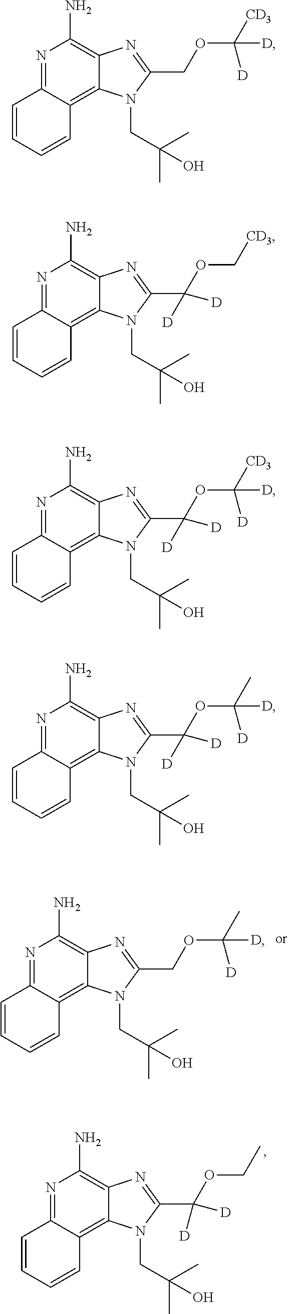
or a pharmaceutically acceptable salt thereof.

12. The combination of claim 10, wherein said immunotherapeutic is: 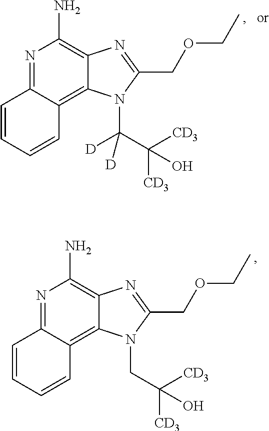 [SMILES: [2H]C([2H])([2H])C(O)(C([2H])([2H])[2H])C([2H])([2H])n1c(COCC)nc2c(N)nc3ccccc3c21 ; [2H]C([2H])([2H])C(O)(Cn1c(COCC)nc2c(N)nc3ccccc3c21)C([2H])([2H])[2H]]
or a pharmaceutically acceptable salt thereof.

13. The combination of claim 1, wherein said targeted therapeutic is capable of binding to a tumor cell specifically or preferably in comparison to a non-tumor cell.

14. The combination of claim 1, wherein said tumor cell is of a carcinoma, a sarcoma, a lymphoma, a myeloma, or a central nervous system cancer.

15. The combination of claim 1, wherein said targeted therapeutic is capable of binding to a tumor antigen specifically or preferably in comparison to a non-tumor antigen.

16. The combination of claim 1, wherein said tumor antigen is selected from the group consisting of: 5T4, AGS-5, AGS-16, Angiopoietin 2, B7.1, B7.2, B7DC, B7H1, B7H2, B7H3, BT-062, BTLA, CAIX, Carcinoembryonic antigen, CTLA4, Cripto, ED-B, ErbB1, ErbB2, ErbB3, ErbB4, EGFL7, EpCAM, EphA2, EphA3, EphB2, FAP, Fibronectin, Folate Receptor, Ganglioside GM3, GD2, glucocorticoid-induced tumor necrosis factor receptor (GITR), gp100, gpA33, GPNMB, ICOS, IGF1R, Integrin αv, Integrin αvβ, KIR, LAG3, Lewis Y, Mesothelin, c-MET, MN Carbonic anhydrase IX, MUC1, MUC16, Nectin-4, NKGD2, NOTCH, OX40, OX40L, PD-1, PD-L1, PSCA, PSMA, RANKL, ROR1, ROR2, SLC44A4, Syndecan-1, TACI, TAG-72, Tenascin, TIM3, TRAILR1, TRAILR2, VEGFR-1, VEGFR-2, VEGFR-3, TIGIT, ILT2, ILT4, SIRPα, TROP2, Claudin18.2, CEACAM5, CD2, CD19, CD20, CD22, CD27, CD33, CD37, CD38, CD40, CD44, CD47, CD52, CD56, CD70, CD73, CD79, CD133, CD137, CD206 and variants thereof.

17. The combination of claim 1, wherein said targeted therapeutic comprises an immunoglobulin, a protein, a peptide, a small molecule, a nanoparticle, or a nucleic acid.

18. The combination of claim 1, wherein said targeted therapeutic comprises an antibody, or a functional fragment thereof.

19. The combination of claim 1, wherein said targeted therapeutic comprises a Fab, Fab′, F(ab′)2, single domain antibody, T and Abs dimer, Fv, scFv, dsFv, ds-scFv, Fd, linear antibody, minibody, diabody, bispecific antibody fragment, bibody, tribody, sc-diabody, kappa (lamda) body, BiTE, DVD-Ig, SIP, SMIP, DART, or an antibody analogue comprising one or more CDRs.

20. A method for treating a tumor or abnormal cell proliferation in a subject that is in need of such treatment, comprising administering to said subject the combination of one of claims 1-19.

21. The method of claim 20, wherein said abnormal cell proliferation comprises a pre-cancerous lesion.

22. The method of claim 21, wherein said abnormal proliferation is of cancer cells.

23. The method of claim 22, wherein said cancer is selected from the group consisting of: breast cancer, colorectal cancer, diffuse large B-cell lymphoma, endometrial cancer, follicular lymphoma, gastric cancer, glioblastoma, head and neck cancer, hepatocellular cancer, lung cancer, melanoma, multiple myeloma, ovarian cancer, pancreatic cancer, prostate cancer, and renal cell carcinoma.

24. The method of claim 20, comprising administering to said subject a formulation comprising said immunotherapeutic in a dose of between about 0.0005 mg/kg, 0.0006 mg/kg, 0.0007 mg/kg, 0.0008 mg/kg, 0.0009 mg/kg, 0.001 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, 0.01 mg/kg, 0.02 mg/kg, 0.025 mg/kg, 0.05 mg/kg, 0.075 mg/kg, 0.1 mg/kg, 0.125 mg/kg, 0.15 mg/kg, 0.175 mg/kg, 0.2 mg/kg, 0.215 mg/kg, 0.25 mg/kg, 0.5 mg/kg, 0.75 mg/kg, 1 mg/kg, 1.25 mg/kg, 1.5 mg/kg, 1.75 mg/kg, 2 mg/kg, 2.25 mg/kg, to about 2.5 mg/kg all inclusive, twice daily, once daily, once every two, three, four, five or six days, or once, twice, or three times per week.

25. The method of claim 20, comprising administering to said subject a formulation comprising said immunotherapeutic in a dose of between about 0.0005 mg/kg, to about 0.0006 mg/kg, 0.0007 mg/kg, 0.0008 mg/kg, 0.0009 mg/kg, 0.001 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, 0.01 mg/kg, 0.02 mg/kg, 0.025 mg/kg, 0.05 mg/kg, 0.075 mg/kg, 0.1 mg/kg, 0.125 mg/kg, 0.15 mg/kg, 0.175 mg/kg, 0.2 mg/kg, 0.215 mg/kg, 0.25 mg/kg, 0.5 mg/kg, 0.75 mg/kg, 1 mg/kg, 1.25 mg/kg, 1.5 mg/kg, 1.75 mg/kg, 2 mg/kg, 2.25 mg/kg, or 2.5 mg/kg all inclusive, twice daily, once daily, once every two, three, four, five or six days, or once, twice, or three times per week.

26. The method of claim 20, comprising administering to said subject a formulation comprising said immunotherapeutic in a dose of less than or about 0.0005 mg/kg, 0.0006 mg/kg, 0.0007 mg/kg, 0.0008 mg/kg, 0.0009 mg/kg, 0.001 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, 0.01 mg/kg, 0.02 mg/kg, 0.025 mg/kg, 0.05 mg/kg, 0.075 mg/kg, 0.1 mg/kg, 0.125 mg/kg, 0.15 mg/kg, 0.175 mg/kg, 0.2 mg/kg, 0.215 mg/kg, 0.25 mg/kg, 0.5 mg/kg, 0.75 mg/kg, 1 mg/kg, 1.25 mg/kg, 1.5 mg/kg, 1.75 mg/kg, 2 mg/kg, 2.25 mg/kg, or 2.5 mg/kg, twice daily, once daily, once every two, three, four, five or six days, or once, twice, or three times per week.

27. The method of claim 20, wherein said administration is orally, sublingually, intravenously, intramuscularly, subcutaneously, or intratumorally.

28. The method of claim 20, wherein said immunotherapeutic in said subject has a local concentration that is between about 0.005 μg/ml to about 12 μg/ml.

29. The method of claim 20, wherein said immunotherapeutic in said subject has a local concentration that is between about 0.05 μg/ml, 0.1 μg/ml, 0.15 μg/ml, 0.2 μg/ml, 0.3 μg/ml, or 0.4 μg/ml, to about 0.5 μg/ml.

30. A kit, comprising the combination of claim 1.
